(19) United States
(12) Patent Application Publication
(10) Pub. No.: US 2022/0184191 A1
(43) Pub. Date: Jun. 16, 2022

(54) Nucleic acid vaccination using neo-epitope encoding constructs

(71) Applicant: Evaxion Biotech A/S, Hørsholm (DK)

(72) Inventors: Birgitte Rønø, Hørsholm (DK); Jens Kringelum, Hørsholm (DK); Anders Bundgård Sørensen, Hørsholm (DK); Thomas Trolle, Hørsholm (DK)

(21) Appl. No.: 17/437,940

(22) PCT Filed: Mar. 11, 2020
(86) PCT No.: PCT/EP2020/056541

§ 371 Date: Sep. 10, 2021

(30) Foreign Application Priority Data
Mar. 11, 2019 (EP) 19161919.6
Oct. 10, 2019 (EP) 19202364.6

(51) Int. Cl. A61K 39/00; A61K 39/39

(52) U.S. Cl. CPC A61K 39/0011; A61K 2039/6093; A61K 2039/53; A61K 39/39

(57) ABSTRACT

Compositions and methods for DNA vaccination that target cancer. The method for anti-cancer vaccination using a plasmid based vaccine comprises regions encoding neo-epitopes and comprising amphiphilic block copolymers such as poloxamer and poloxamine agents.

### FIELD OF THE INVENTION

The present invention relates to cancer therapy, in particular cancer immunotherapy. In particular, the present invention relates to methods and products for treating cancer by nucleic acid vaccination.

### BACKGROUND OF THE INVENTION

Treatment of malignant neoplasms in patients has traditionally focussed on eradication/removal of the malignant tissue via surgery, radiotherapy, and/or chemotherapy using cytotoxic drugs in dosage regimens that aim at preferential killing of malignant cells compared to killing of non-malignant cells.

In addition to the use of cytotoxic drugs, more recent approaches have focussed on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance proposed by Burnet and Thomas suggested that lymphocytes recognize and eliminate autologous cells—including cancer cells—that exhibit altered antigenic determinants, and it is today generally accepted that the immune system to a high degree contributes to control of primary tumour growth and eliminates metastasis. Nevertheless, immunosurveillance is not 100% effective and it is a continuing task to device cancer therapies where the immune system's ability to eradicate cancer cells is sought improved/stimulated.

One approach has been to induce immunity against cancer associated antigens, but even though this approach has the potential of being promising, it suffers the same drawback as antibody therapy that only a limited number of antigens can be addressed.

Many if not all tumours express mutations. These mutations potentially create new targetable antigens (neo-antigens), which are potentially useful in specific T cell immunotherapy if it is possible to identify the neo-antigens and their antigenic determinants within a clinically relevant time frame. Since it with current technology is possible to fully sequence the genome of cells and to analyse for existence of altered or new expression gene products, it is possible to design personalized vaccines based neo-antigens. However, attempts at providing satisfactory clinical end-points have as today largely failed or are still in the early non-conclusive stages.

One mode of vaccination that has been investigated in detail since the early 1990'ies is nucleic acid vaccination (also termed DNA vaccination), where DNA is administered in a non-viral plasmid form to somatic cells of a mammal leading to expression of the inserts comprised in the plasmid; in DNA vaccination the encoded material is immunogenic polypeptide(s), which upon production by the somatic cells will be able to induce an immune response. This approach is appealing as it avoids the need of producing the protein immunogen in clinical grade purity using expensive recombinant expression systems. However, it has proven difficult to obtain expression levels from the DNA administered, which are high enough to effect satisfactory immune responses in humans.

There is hence an existing need for provision of anti-cancer vaccines, in particular nucleic acid vaccines, that can effectively target neo-antigens and induce clinically significant immune responses in vaccinated human beings.

### OBJECT OF THE INVENTION

It is an object of embodiments of the invention to provide methods and products for use in nucleic acid vaccination in order to treat or ameliorate cancer in larger mammals such as humans.

### SUMMARY OF THE INVENTION

It has been found by the present inventor(s) that immunization of mice with dosages of DNA vaccine plasmids encoding identified neo-epitopes of the cancer, is able to provide protective immunity against the cancer; dosages further translate into human dosages that would be clinically acceptable.

So, in a first aspect the present invention relates to a method of inducing a therapeutic or ameliorating immune response against a malignant neoplasm in a patient, wherein the cells of the malignant neoplasm express genetic material that encode neo-epitope containing polypeptides, the method comprising administering to the patient at least one effective dosage of a composition comprising

1) at least one expression vector, which comprises nucleic acid(s) encoding at least one polypeptide, which exhibits one or more neo-epitopes of the malignant neoplasm, and

2) an amphiphilic block co-polymer comprising blocks of poly(ethylene oxide) and polypropylene oxide), and

3) a pharmaceutically acceptable carrier or diluent,

whereby somatic cells in the patient are brought to express the nucleic acid(s) encoding the at least one polypeptide.

In a second aspect the invention relates to a composition comprising

1) at least one expression vector, which comprises nucleic acid(s) encoding at least one polypeptide, which exhibits one or more neo-epitopes of a malignant neoplasm, and

2) an amphiphilic block co-polymer comprising blocks of poly(ethylene oxide) and polypropylene oxide), and

3) a pharmaceutically acceptable carrier or diluent,

i.e. a composition identical to the one administered in the method of the first aspect of the invention.

In third and fourth aspects, the invention relates to a composition of the second aspect 1) for use as a medicament, and 2) for use in a method of the first aspect, respectively.

### LEGENDS TO THE FIGURE

FIG. 1: Plasmid map of pVAX1.

Details are provided in the examples and SEQ ID NO: 1.

FIG. 2: Plasmid map of pVAX1 S16A.

Details are provided in the examples.

FIG. 3: Plasmid map of pVAX1 S16B.

Details are provided in the examples.

FIG. 4: Diagram shown detection of C22 MHC I multimers.

The graph shows the frequency of murine CD8+ T cells reactive with the C22 peptide upon vaccination of mice with experimental DNA vaccines.

FIG. 5: Plasmid map showing preferred position of ISS in pTVG4.

FIG. 6: Tumour volume (area under curve, AUC) in mice vaccinated with Lutrol and vaccine plasmids.

See example 1 for details.

FIG. 7: Tumour volume (area under curve, AUC) in mice vaccinated with test vaccines.

See Example 2 for details.

FIG. 8: C22 tetramer staining of CD8+ T cells isolated from vaccinated mice.

See Example 2 for details.

FIG. 9: Tumour sizes (area-under-curve, AUC), in mice vaccinated with test vaccines.

See Example 3 for details.

FIG. 10: C22 tetramer staining of CD8⁺ T cells isolated from vaccinated mice.

See Example 3 for details.

FIG. 11: Tumour sizes (area-under-curve, AUC), in mice vaccinated with test vaccines.

See Example 4 for details.

*: p<0.05 (Kruskal-Wallis test)

**: p<0.01 (Kruskal-Wallis test)

***: p<0.001 (Kruskal-Wallis test)

****: p<<0.001 (Kruskal-Wallis test)

FIG. 12: C22 tetramer staining of CD8⁺ T cells isolated from vaccinated mice.

See Example 4 for details.

FIG. 13: Graph showing reactive T cells capable of producing multiple cytokines (TNFα and IFNγ).

A: Data from CD8⁺ cells.

B: Data from CD4⁺ cells.

### DETAILED DISCLOSURE OF THE INVENTION

### Definitions

A “PEO-PPO” amphiphilic block co-polymer is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (“poloxamers”), PPO-PEO-PPO, (PEO-PPO-)₄ED (a “poloxamine”), and (PPO-PEO-)₄ED (a “reverse poloxamine”), where “ED” is a ethylenediaminyl group.

A “poloxamer” is a linear amphiphilic block copolymer constituted by one block of poly(ethylene oxide) (“PEO”) coupled to one block of poly(propylene oxide) (“PPO”) coupled to one block of PEO, i.e. a structure of the formula EOₐ-PO_{b}-EOₐ, where EO is ethylene oxide, PO is propylene oxide, a is an integer ranging between 2 and 130, and b is an integer ranging between 15 and 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of M_{w}≈1800 (corresponding to b≈31 PPO) and approximately 80% (w/w) of PEO (corresponding to a≈82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) are X-shaped block copolymers that bear four PEO-PPO arms connected to a central ethylenediamine via bonds between the free OH groups in the PEO-PPO- groups and the primary amine groups in ethylenediamine, and “reverse poloxamine” are likewise X-shaped block copolymers that bear four PPO-PEO arms connected to a central ethylenediamine via bonds between the free OH groups in the PPO-PEO- groups and the primary amine groups in ethylenediamine.

A “cancer specific” antigen is an antigen, which does not appear as an expression product in an individual's non-malignant somatic cells, but which appears as an expression product in cancer cells in the individual. This is in contrast to “cancer-associated” antigens, which also appear—albeit at low abundance—in normal somatic cells, but are found in higher levels in at least some tumour cells.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“CAF09” (Cationic Adjuvant Formulation 09) is an immunologic adjuvant liposome formulation, which comprises the quaternary ammonium surfactant N,N-dimethyl-N,N-dioctadecylammonium (DDA), a synthetic 3-hydroxy-2-tetradecyl-octadecanoic acid-2,3-dihydroxypropyl ester (monomycolyl glycerol, “MMG”), which acts as a ligand for C-type lectin receptors (CLRs), and Polyinosinic-polycytidylic acid (sodium salt) (“poly-IC” or “poly(I:C)”), which acts as a ligand for toll-like receptors (“TLRs”). A number of CAF family adjuvants, including CAF09, is disclosed in detail in US 2014/0112979 and in US 2016/0228528. The relative amounts (w:w:w) of DDA:MMG:Poly(I:C) are 5:1:1.

“CAF09b” is a version of CAF09 with the relative amount of poly(I:C) reduced to about ¼ of the amount disclosed in US 2014/0112979: in CAF09, the relative amounts (w:w:w) of DDA:MMG:poly(I:C) are thus 20:4:1 with a typical human dose containing 625 μg DDA, 125 μg DDA, and 31.25 μg poly(I:C), respectively.

A “neo-epitope” is an antigenic determinant (typically an MHC Class I or II restricted epitope), which does not exist as an expression product from normal somatic cells in an individual due to the lack of a gene encoding the neo-epitope, but which exists as an expression product in mutated cells (such as cancer cells) in the same individual. As a consequence, a neo-epitope is from an immunological viewpoint truly non-self in spite of its autologous origin and it can therefore be characterized as a tumour specific antigen in the individual, where it constitutes an expression product. Being non-self, a neo-epitope has the potential of being able to elicit a specific adaptive immune response in the individual, where the elicited immune response is specific for antigens and cells that harbour the neo-epitope. Neo-epitopes are on the other hand specific for an individual as the chances that the same neo-epitope will be an expression product in other individuals is minimal. Several features thus contrast a neo-epitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neo-peptide” is a peptide (i.e. a polyamino acid of up to about 50 amino acid residues), which includes within its sequence a neo-epitope as defined herein. A neo-peptide is typically “native”, i.e. the entire amino acid sequence of the neo-peptide constitutes a fragment of an expression product that can be isolated from the individual, but a neo-peptide can also be “artificial”, meaning that it is constituted by the sequence of a neo-epitope and 1 or 2 appended amino acid sequences of which at least one is not naturally associated with the neo-epitope. In the latter case the appended amino acid sequences may simply act as carriers of the neo-epitope, or may even improve the immunogenicity of the neo-epitope (e.g. by facilitating processing of the neo-peptide by antigen-presenting cells, improving biologic half-life of the neo-peptide, or modifying solubility).

A “neo-antigen” is any antigen, which comprises a neo-epitope. Typically, a neo-antigen will be constituted by a protein, but a neo-antigen can, depending on its length, also be identical to a neo-epitope or a neo-peptide.

The term “amino acid sequence” is the order in which amino acid residues, connected by peptide bonds, lie in the chain in peptides and proteins. Sequences are conventionally listed in the N to C terminal direction.

A “linker” is an amino acid sequence, which is introduced between two other amino acid sequences in order to separate them spatially. A linker may be “rigid”, meaning that it does substantially not allow the two amino acid sequences that it connects to move freely relative to each other. Likewise, a “flexible” linker allows the two sequences connected via the linker to move substantially freely relative to each other. In encoded expression products that contain more than one neo-epitope, both types of linkers are useful. However, one particular interesting linker useful in the present invention has the 12 amino acid residue sequence AEAAAKEAAAKA (SEQ ID NO: 9).

Other linkers of interest, which can be encoded by an expression vector used in the invention, are listed in the following table:

| Type | Name | Sequence |
|---|---|---|
| Flexible | FS | GSGGGA (SEQ ID NO: 3) |
| Flexible | FL | GSGGGAGSGGGA (SEQ ID NO: 4) |
| Flexible | FV1 | GSGGGAGSGGGAGSGGGA (SEQ ID NO: 5) |
| Flexible | FV2 | GSGGGAGSGGGAGSGGGAGSGGGA (SEQ ID |
| | | NO: 6) |
| Flexible | FM | GENLYFQSGG (SEQ ID NO: 7) |
| Rigid | RL1 | KPEPKPAPAPKP (SEQ ID NO: 8) |
| Rigid | RL2 | AEAAAKEAAAKA (SEQ ID NO: 9) |
| Rigid | RM | SACYCELS (SEQ ID NO: 10) |
| Flexible | | SGGGSSGGGS (SEQ ID NO: 22) |
| Flexible | | GGGGSGGGGS (SEQ ID NO: 23) |
| Flexible | | SSGGGSSGGG (SEQ ID NO: 24) |
| Flexible | | GGSGGGGSGG (SEQ ID NO: 25) |
| Flexible | | GSGSGSGSGS (SEQ ID NO: 26) |

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T helper lymphocyte response against the combined substance constituted by the immunogen and the immunogenic carrier, and this in turn provides for improved responses against the immunogen by B lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the antigens bound to MHC molecules are being recognized by T cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or T-cell receptors (TCRs) that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

### Embodiments of the First Aspect of the Invention

In the method disclosed herein, a therapeutic or ameliorating immune response against a malignant neoplasm is induced in a patient (preferably a human), wherein the cells of the malignant neoplasm express genetic material that encode neo-epitope containing polypeptides. The method comprises administering to the patient at least one effective dosage of a composition comprising

1) at least one expression vector, which comprises nucleic acid(s) encoding at least one polypeptide, which exhibits one or more neo-epitopes of the malignant neoplasm, and

2) an amphiphilic block co-polymer comprising blocks of poly(ethylene oxide) and polypropylene oxide), and

3) a pharmaceutically acceptable carrier, diluent, or excipient,

whereby somatic cells in the patient are brought to express the nucleic acid(s) encoding the at least one polypeptide.

The expression vector is typically and preferably comprised in or constituted by a plasmid, but other expression vectors can be employed. The composition of the present invention and its content of the amphiphilic block co-polymer aims at ensuring delivery of “naked” DNA to cells, i.e. a DNA expression vector, which is not part of a bacterium or virus that would be able to effect introduction of the expression vector into the target cells. A vector useful in the present compositions and methods can thus be circular or linear, single-stranded or double stranded and can in addition to a plasmid also be e.g. a cosmid, mini-chromosome or episome.

Each coding (and expressible) region can be present on the same or on separate vectors; however, it is to be understood that one or more coding regions can be present on a single vector, and these coding regions can be under the control of a single or multiple promoters. This means that the expression vector can encode a separate peptide expression product for each encoded neo-epitope or that the expression vector can encode a plurality of peptide expression products, where at least some exhibit(s) several encoded neo-epitopes, of which at least some optionally are separated by peptide linkers.

In other words, in some cases only one single expression vector is administered and expressed, and this expression vector may encode a plurality of separate proteinaceous expression products or as few as 2 or even one single expression product—in this context it is only relevant whether the encoded neo-epitopes are satisfactorily presented to the immune system and the choice of their presence in separate on combined expression products is therefore of minor relevance. In preferred embodiments, the expression vector expresses at least or about 5, such as at least or about 10, at least or about 15, at least or about 20, at least or about 25, at least or about 30 proteinaceous expression products. Higher numbers are contemplated and the limit is primarily set by the number of neo-epitopes it is possible to identify from a particular neoplasm. It goes without saying that the number of encoded neo-epitopes in the expression vector(s) cannot exceed the number of neo-epitopes found in the relevant malignant tissue.

The use of peptide linkers to separate encoded neo-epitope expression products provides spatial separation between epitopes in the expression product. This can entail several advantages: linkers can ensure that each neo-epitope is presented in an optimized configuration to the immune system, but use of appropriate linkers can also minimize the problem that irrelevant immune responses are directed against “junctional epitopes” which emerge in the regions constituted by the C-terminal end of one neo-epitope and the adjacent N-terminal end of the next neo-epitope in a multi-epitope containing expression product.

Encoded peptide linkers can be either “flexible” or “rigid”, cf. the definition above, where preferred encoded linkers are set forth. Also, it is envisaged that the linker(s) used in the invention in some embodiments can be cleavable, that is, include (a) recognition site(s) for endopeptidase(s), e.g. endopeptidases such as furin, caspases, cathepsins etc.

The neo-epitopes encoded by the expression vector can be identified in a manner known per se: “deep sequencing” of the genome of the malignant cells and of the genome of healthy cells in the same individual or a standard healthy genome can identify expressed DNA sections that provide for potentially immunogenic expression products unique to the malignant cells. The identified DNA sequences can thereafter be codon-optimized (typically for expression by human cells) and included in the expression vector—either as separate expression regions of as part of larger chimeric constructs.

In order to optimize the identification and selection of the neo-epitopes that are to be expressed by the vector, any of the prediction methods available for this purpose are in practice useful. One example of state of the art prediction algorithm is NetMHCpan-4.0 (www.cbs.dtu.dk/services/NetMHCpan/; Jurtz V et al., J Immunol (2017), ji1700893; DOI: 10.4049/jimmunol.1700893). This method is trained on a combination of classical MS derived ligands and pMHC affinity data. Another example is NetMHCstabpan-1.0 (www.cbs.dtu.dk/services/NetMHCstabpan/; RasmussenM et al., Accepted for J of Immunol, June 2016). This method is trained on a dataset of in vitro pMHC stability measurement using an assay where each peptide is synthesized and complexed to the MHC molecule in vitro. No cell processing is involved in this assay and the environment where the pMHC stability is measured is somewhat artificial. The method in general is less accurate than NetMHCpan-4.0. U.S. Pat. No. 10,055,540 describes a method for identification of neo-epitopes using classical MS detected ligands. Other patent application publications using similar technology are WO 2019/104203, WO 2019/075112, WO 2018/195357 (MHC Class II specific), and WO 2017 106638. Finally, MHCflurry:

www-sciencedirect-com.proxy.findit.dtu.dk/science/article/pii/S2405471218302321 is like NetMHCpan trained on MS detected ligand data and pMHC affinities. A peptide-MHC Class II interaction prediction method is also disclosed in a recent publication Garde C et al., Immunogenetics, DOI: doi.org/10.1007/s00251-019-01122-z. In this publication, naturally processed peptides eluted from MHC Class II are used as part of the training set and assigned the binding target value of 1 if verified as ligands and 0 if negative.

Generally, these prediction systems employ artificial neural networks (ANNs): ANNs can identify non-linear correlations: Quantification of non-linear correlations is not an easy task, since it is difficult to calculate by simple calculation. This is primarily due to non-linear correlations described with more parameters than linear correlations and probably first appear when all features are considered collectively. Hence it is needed to take all features into account in order to catch the dependency across features.

In order to further improve the likelihood that the selected encoded neo-epitopes provide for an effective immune response, use can preferably be made of the technology disclosed in European patent application nos: 19197295.9 and 19197306.4, both filed on 13 Sep. 2019. These applications disclose technology, which enables that stability of binding between peptides and MHC molecules can be determined and which enables that stability of MHC binding of neo-epitopes is determined as part of the neo-epitope detection and selection. In brief, the data obtained from stability determinations are e.g. used as part of the training set for an ANN, and the ANN can subsequently rank identified peptides according to their predicted binding stabilities towards relevant MHC molecules.

When a nucleic acid vaccine is administered to a patient, the corresponding gene product (such as a desired antigen) is produced in the patient's body. In some embodiments, nucleic acid vaccine vectors that include optimized recombinant polynucleotides can be delivered to human to induce a therapeutic or prophylactic immune response.

Plasmid and other naked DNA vectors are typically more efficient for gene transfer to muscle tissue. The potential to deliver DNA vectors to mucosal surfaces by oral administration has also been reported and DNA plasmids have been utilized for direct introduction of genes into other tissues than muscle. DNA vaccines have been introduced into animals primarily by intramuscular injection, by gene gun delivery, by jet injection (using a device such as a Stratis® device from PharmaJet), or by electroporation; each of these modes of administration apply to the presently disclosed method. After being introduced, the plasmids are generally maintained episomally without replication. Expression of the encoded proteins has been shown to persist for extended time periods, providing stimulation of both B and T cells.

In determining the effective amount of the vector to be administered in the treatment method disclosed herein, the physician evaluates vector toxicities, vaccine-induced adverse events, progression of the cancer to be treated, and the production of anti-vector antibodies, if any. Administration can be accomplished via single or divided doses and typically as a series of time separated administrations. In the methods disclosed herein, the effective human dose per immunization in a time-separated series is between 10 μg and 500 mg, with dosages between 100 μg and 25 mg of the expression vector being preferred. That is, in the practice of the method disclosed herein dosages of between 1 and 20 mg in humans are typically used, and dosages are normally between 0.5 and 15 mg, between 1 and 10 mg, and between 2 and 8 mg, and particular interesting dosages are of about 0.5, about 1, about 2, about 3, about 4, about 5, about 6, about 7 and about 8 mg.

A series of immunizations with effective dosages will typically constitute a series of 2, 3, 4, 5, 6, or more dosages. Multiple (e.g. >6) dosages may for instance be relevant in order to keep a malignant neoplasm in check for a prolonged period and in such a situation the exact choice of encoded neo-epitopes in the vaccine vector can be changed over time in response to changes in the genome and proteome of the malignant cells. When and if new neo-epitopes are produced by the malignant cells these can conveniently be included as targets for the vaccine.

The vaccine used in the method disclosed herein comprises one or more expression vectors; for instance, the vaccine may comprise a plurality of expression vectors each capable of autonomous expression of a nucleotide coding region in a mammalian cell to produce at least one immunogenic polypeptide. An expression vector often includes a eukaryotic promoter sequence, such as the nucleotide sequence of a strong eukaryotic promoter, operably linked to one or more coding regions. The compositions and methods herein may involve the use of any particular eukaryotic promoter, and a wide variety are known; such as a CMV or RSV promoter. The promoter can be heterologous with respect to the host cell. The promoter used may be a constitutive promoter. The promoter used may include an enhancer region and an intron region to improve expression levels, such as is the case when using a CMV promoter.

Numerous plasmids known in the art may be used for the production of nucleic acid vaccines. Suitable embodiments of the nucleic acid vaccine employ constructs using the plasmids VR1012 (Vical Inc., San Diego Calif.), pCMVI.UBF3/2 (S. Johnston, University of Texas), pTVG4 (Johnson et al., 2006, Vaccine 24(3); 293-303), pVAX1 (Thermo Fisher Scientific, see above and the Examples below), or pcDNA3.1 (InVitrogen Corporation, Carlsbad, Calif.) as the vector.

In addition, the vector construct can according to the present invention advantageously contain immunostimulatory sequences (ISS). The aim of using such sequences in the vaccine vector is to enhance T-cell response towards encoded neo-epitopes, in particular Th1 cell responses, which are elicited by adjuvants that incorporate agonists of the toll-like receptors TLR3, TLR7-TLR8, and TLR9. and/or cytosolic RNA receptors such as, but not limited to, RIG-1, MDA5 and LGP2 (Desmet et al. 2012. Nat. Rev. Imm. 12(7), 479-491)

One possibility of employing ISS is to mimic a bacterial infection activating TLR9 by stimulating with non-methylated CG-rich motifs (so-called CpG motifs) of six bases with the general sequence NNCGNN (which have a 20-fold higher frequency in bacterial DNA than in mammalian DNA) either as directly administered small synthetic DNA oligos (ODNs), which contain partially or completely phosphorothioated backbones, or by incorporating the CpG motifs in the DNA vector backbone. Immunostimulatory CpGs can be part of the DNA backbone or be concentrated in an ISS where the CpG sequence(s) typically will be positioned between the stop codon in the neo-epitope coding sequence and the poly-A tail encoding sequence (i.e. the ISS is located between the stop codon and the polyadenylation signal). However, since CpG sequences exert an effect irrespectively of their position in a longer DNA molecule, their position could in principle be anywhere in the vaccine vector as long as the presence of the CpG motif does not interfere with the vector's ability to express the coding regions of the vaccine antigen.

If present in the vaccine as separate ODNs, where the ODNs function as immunological adjuvants, CpG motif containing oligonucleotides are typically to be co-administered/formulated together with the DNA vaccine by the selected delivery technology and will typically constitute hexamers or longer multimers of DNA comprising the sequence NNCGNN or the reverse complementary sequence. Useful ODNs for this purpose are e.g. commercially available from InvivoGen, 5 Rue Jean Rodier, F-31400, Toulouse, France, which markets a range of Class A, B, and C ODNs. Examples are:

ODN1585
SEQ ID NO: 27
(5′-ggGGTCAACGTTGAgggggg-3′),
ODN2216
SEQ ID NO: 28
(5′-ggGGGACGATCGTCgggggg-3′),
ODN2336
SEQ ID NO: 29
(5′-gggGACGACGTCGTGgggggg-3′),
ODN1668
SEQ ID NO: 30
(5′-tccatgacgttcctgatgct-3′),
ODN1826
SEQ ID NO: 31
(5′-tccatgacgttcctgacgtt-3′),
ODN2006
SEQ ID NO: 32
(5′-tcgtcgttttgtcgttttgtcgtt-3′),
ODN2007
SEQ ID NO: 33
(5′-tcgtcgttgtcgttttgtcgtt-3′),
ODNBW006
SEQ ID NO: 34
(5′-tcgacgttcgtcgttcgtcgttc-3′),
ODN D-SL01
SEQ ID NO: 35
(5′-tcgcgacgttcgcccgacgttcggta-3′),
ODN2395
SEQ ID NO: 36
(5′-tcgtcgttttcggcgcgcgccg-3′),
ODN M362
SEQ ID NO: 37
(5′-tcgtcgtcgttcgaacgacgttgat-3′),
ODN D-SL03
SEQ ID NO: 38
(5′-tcgcgaacgttcgccgcgttcgaacgcgg-3′),

In these 12 ODNs, upper case nucleotides are phosphodiesters, lower case nucleotides are phosphorothioates, and underlining denotes palindromic sequences.

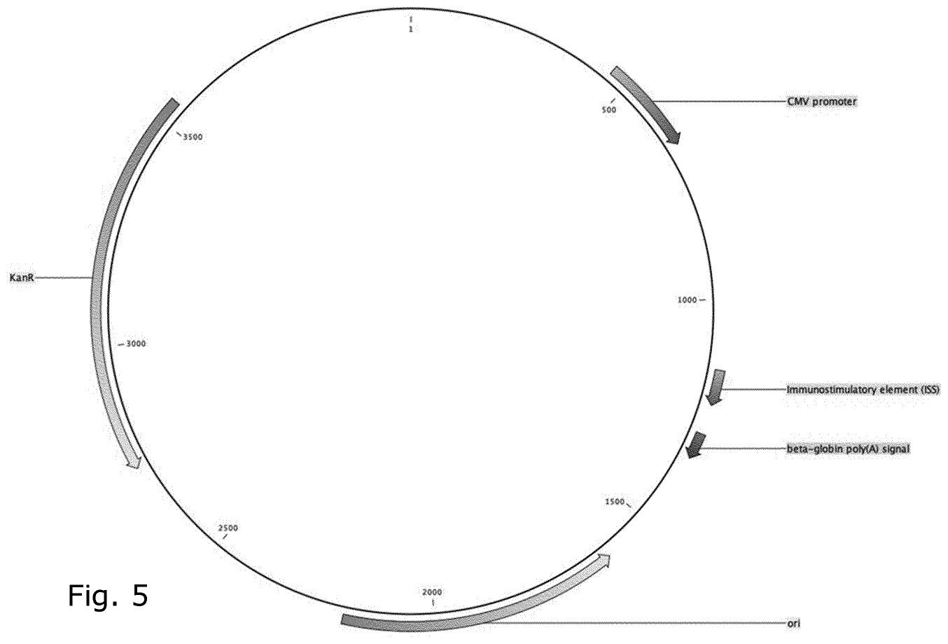

When CpG sequences are present in the plasmid backbone (which thereby become “self-adjuvating”), any number of possible NNGCNN sequences can according to the invention be present, either as identical sequences or in the form of non-identical sequences of the CpG motif, or in the form of palindromic sequences that can form stem-loop structures. For instance, the following CpG motifs are of interest: AACGAC and GTCGTT, but also CTCGTT, and GCTGTT. An example the use of such CpG encoding sequences is the following sequence excerpt from the commercially available pTVG4 vaccine vector backbone (which is schematically shown in its entirety in FIG. 5 and in SEQ ID NO: 40): . . . agatctaacaacaaaacaacaaaacaacaaggcgccagatctggcgtttcgttttgtcgttttgtcgttagatct . . . (SEQ ID NO: 41), where the underlined nucleotides constitute the CpGs that are present in the pTVG4 plasmid vector sequence.

Another possibility is to mimic an RNA viral infection to activate TLR3 by adding a double stranded(ds) RNA either as synthetic RNA oligos such as Poly I:C (polyinosinic-polycytidylic acid), Poly I:CU12 (uridine-substituted Poly I:C), or in the form of synthetic RNA oligonucleotides (ORNs); addition of these RNA molecules to the vaccine is, as for the ODN approach, a way of obtaining an adjuvant effect. Alternatively, the dsRNA can be encoded in the DNA vector backbone, which will be transcribed into RNA after vaccination—in this case the DNA vaccine hence encodes the immunological adjuvant. This approach can include DNA sequences that encode hairpin RNA with lengths of up to 100 base pairs, where the sequence is unspecific. Also the DNA can simultaneously include ODNs and encode ORNs of known sequences; the DNA can thus both be transcribed into a double stranded RNA capable of activating TLR3 and/or cytosolic RNA receptors such as RIG-1, MDAS, and LGP2 while comprising an ODN to activate TLR9. Examples of specific DNA sequences that include/encode immune stimulating CpG and dsRNA are for instance 5′-GGTGCATCGA TGCAGGGGGG-3′ (SEQ ID NO: 41) and 5′-GGTGCATCGA TGCAGGGGGG TATATATATA TTGAGGACAG GTTAAGCTCC CCCCAGCTTA ACCTGTCCTT CAATATATA TATA-3′ (SEQ ID NO: 42) (ref: Wu et al., 2011, Vaccine 29(44): 7624-30).

When ISS are present in the DNA vaccine vector, it is possible—and advantageous—to combine the approach of using CpG motifs to activate TLR9 with the presence of coding sequences for immune stimulating RNA to activate TLR3 and /or cytosolic RNA receptors such as RIG-1, MDAS, and LGP2; cf. Grossmann C et al. 2009, BMC. Immunology 10:43 and Desmet et al. 2012. Nat. Rev. Imm. 12(7), 479-491. Likewise, incorporation of ORNs and ODNs in the vaccine as separate adjuvants (alone or in combination) may be combined with the incorporation of ISS of both types in the DNA vaccine vector.

As is the case for the CpG motifs, the DNA encoding the immune stimulatory RNA ISS will preferably be present between the stop codon and the polyadenylation signal but can be present in any part of the vector as long as this does not impair the production of the intended polypeptide expression product.

In particularly important embodiments, ISS is/are comprised in the vaccine compositions, and in particular important embodiments this is achieved by incorporating an immunologically active and pharmaceutically acceptable amount of poly I:C and/or poly IC:U12. Poly I:C is constituted by a mismatched double-stranded RNA (dsRNA) with one strand being a polymer of inosinic acid and the other strand a polymer of cytidylic acid. Poly IC:U12 is a variant of poly I:C where uridine is introduced into the Poly I:C strand. These two substances will in that context function as immunological adjuvants, i.e. substances that themselves do not elicit a specific adaptive immune response, but which enhances the specific adaptive immune response against the vaccine antigen (or in the present case, the encoded antigen).

Poly I:C or poly IC:U12 (such as Ampligen®) will preferably be present in the composition so as to arrive at an administered dosage of between 0.1 and 20 mg per administration of the effective dosage of the expression vector; that is, the amount present in the composition is adjusted so as to arrive at such dosages per administration. Preferably the administered dosage of poly I:C or poly IC:U12 is between 0.2 and 15 mg per administration of the effective dosage of the expression vector, such as between 0.3 and 12, 0.4 and 10 and 0.5 and 8 mg, preferably about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9 and 4.0 mg. Particularly preferred are in the range between 0.5 and 2.0 mg per administration.

The nucleic acid vaccine can also encode a fusion product containing one or more immunogenic polypeptides containing neo-epitopes. Plasmid DNA can also be delivered using attenuated bacteria as delivery system, a method that is suitable for DNA vaccines that are administered orally. Bacteria are transformed with an independently replicating plasmid, which becomes released into the host cell cytoplasm following the death of the attenuated bacterium in the host cell.

DNA vaccines, including the DNA encoding the desired antigen, can be introduced into a host cell in any suitable form including, the fragment alone, a linearized plasmid, a circular plasmid, a plasmid capable of replication, an episome, RNA, etc. Preferably, the gene is contained in a plasmid. In certain embodiments, the plasmid is an expression vector. Individual expression vectors capable of expressing the genetic material can be produced using standard recombinant techniques.

Routes of administration include, but are not limited to, intramuscular, intranasal, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. In other words, the route of administration can be selected from any one of parenteral routes, such as via the intramuscular route, the intradermal route, transdermal route, the subcutaneous route, the intravenous route, the intra-arterial route, the intrathecal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

Typical routes of administration include intramuscular, intraperitoneal, intradermal and subcutaneous injection. Genetic constructs may be administered by means including, but not limited to, traditional syringes, needleless injection devices, “microprojectile bombardment gene guns”, or other physical methods such as electroporation (“EP”), “hydrodynamic method”, or ultrasound. DNA vaccines can be delivered by any method that can be used to deliver DNA as long as the DNA is expressed and the desired antigen is made in the cell.

In some embodiments, a DNA vaccine composition disclosed herein is delivered via or in combination with known transfection reagents such as cationic liposomes, fluorocarbon emulsion, cochleate, tubules, gold particles, biodegradable microspheres, or cationic polymers. Cochleate delivery vehicles are stable phospholipid calcium precipitants consisting of phosphatidyl serine, cholesterol, and calcium; this nontoxic and noninflammatory transfection reagent can be present in a digestive system. Biodegradable microspheres comprise polymers such as poly(lactide-co-glycolide), a polyester that can be used in producing microcapsules of DNA for transfection. Lipid-based microtubes often consist of a lipid of spirally wound two layers packed with their edges joined to each other. When a tubule is used, the nucleic acid can be arranged in the central hollow part thereof for delivery and controlled release into the body of an animal.

A DNA vaccine can also be delivered to mucosal surfaces via microspheres. Bioadhesive microspheres can be prepared using different techniques and can be tailored to adhere to any mucosal tissue including those found in eye, nasal cavity, urinary tract, colon and gastrointestinal tract, offering the possibilities of localized as well as systemic controlled release of vaccines. Application of bioadhesive microspheres to specific mucosal tissues can also be used for localized vaccine action. In some embodiments, an alternative approach for mucosal vaccine delivery is the direct administration to mucosal surfaces of a plasmid DNA expression vector which encodes the gene for a specific protein antigen.

The DNA plasmid vaccines disclosed are formulated according to the mode of administration to be used. Typically, the DNA plasmid vaccines are injectable compositions, they are sterile, and/or pyrogen free and/or particulate free. In some embodiments, an isotonic formulation is preferably used. Generally, additives for isotonicity can include sodium chloride, dextrose, mannitol, sorbitol and lactose. In some embodiments, isotonic solutions such as phosphate buffered saline are preferred; one preferred solution is Tyrode's buffer. In some embodiments, stabilizers include gelatine and albumin. In some embodiments, a stabilizing agent that allows the formulation to be stable at room or ambient temperature for extended periods of time, such as LGS or other poly-cations or poly-anions is added to the formulation.

The DNA vaccine composition disclosed herein comprises a pharmacologically acceptable amphiphilic block co-polymer comprising blocks of poly(ethylene oxide) and polypropylene oxide, which is described in detail in the following:

The amphiphilic block co-polymer is described more generally under the definition heading, but the preferred the amphiphilic block co-polymer is a poloxamer or a poloxamine. Poloxamers only vary slightly with respect to their properties, but preferred are poloxamer 407 and 188, in particular poloxamer 188.

When the amphiphilic block co-polymer is poloxamine, the preferred type is a sequential poloxamine of formula (PEO-PPO)4-ED, where PEO is poly(ethylene oxide), PPO is poly(propylene oxide) and ED is an ethylenediaminyl group. These molecules attain an X-like shape where the PEO-PPO groups protrude from the central ethylenediaminyl group.

Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

When used in the method disclosed herein, the concentration of the amphiphilic block co-polymer in the vaccine composition is between 2 and 5% w/v, such as about 3% w/v.

The third constituent in the composition disclosed herein is the pharmaceutically acceptable carrier, diluent, or excipient, which is preferably in the form of a buffered solution. Parenteral vehicles include sodium chloride solution, Ringer's dextrose and sodium chloride, lactated Ringer's or fixed oils. Intravenous vehicles include fluid and nutrient replenishers, electrolyte replenishers such as those based on Ringer's dextrose, and the like. Preservatives and antimicrobials include antioxidants, chelating agents, inert gases and the like. Preferred preservatives include formalin, thimerosal, neomycin, polymyxin B and amphotericin B.

In preferred embodiments, the buffered solution is the one known as “Tyrode's buffer”, and in preferred embodiments the Tyrode's buffer has the composition 140 mM NaCl, 6 mM KCl, 3 mM CaCl2, 2 mM MgCl2, 10 mM 4-(2-hydroxyethyl)-1-piperazineethanesulfonic acid (Hepes) (preferably at pH 7.4), and 10 mM glucose, or alternatively, 140 mM NaCl, 6 mM KCl, 3 mM CaCl2, 2 mM MgCl2, 10 mM 2-amino-2-(hydroxymethyl)-1,3-propandiol (TRIS) (preferably at pH 7.4), and 10 mM glucose. The concentration of the Tyrodes' buffer (or alternatives) is typically about 35% v/v, but depending on the water content of suspended plasmids, the concentration may vary considerably—since the buffer is physiologically acceptable, it can constitute any percentage of the aqueous phase of the composition.

Other buffers may be utilised, such as 2-amino-2-(hydroxymethyl)-1,3-propandiol (TRIS) or phosphate buffered saline (PBS).

Additional carrier substances may be included and can contain proteins, sugars, etc. Such carriers may be aqueous or non-aqueous solutions, suspensions, and emulsions. Examples of non-aqueous carriers are propylene glycol, polyethylene glycol, vegetable oils such as olive oil, and injectable organic esters such as ethyl oleate. Aqueous carriers include water, alcoholic/aqueous solutions, emulsions or suspensions, including saline.

### 2^{nd} Aspect—Vaccine Composition

The vaccine composition, which constitutes the 2^{nd} aspect of the invention is a composition as described under the first aspect of the invention and in the claims. Hence, each and every feature and consideration that pertains to the composition, which is used in the method disclosed herein, apply mutatis mutandis to the composition of the 2^{nd} aspect of the invention.

3^{rd} and 4^{th} and Related Aspects of the Invention

The two aspects are related in that they concern the composition of the 2^{nd} aspect for a therapeutic use, i.e. the composition of the 2^{nd} aspect of the invention for use as a medicament or for use in a method according of the first aspect of the invention, respectively.

Likewise, the 4^{th} aspect of the invention also covers use of a composition of the 2^{nd} aspect in a method of the 1^{st} aspect as well as use of the components of the composition of the 2^{nd} aspect for use in the preparation of a pharmaceutical composition for use in treatment of a malignant neoplasm.

### Example 1

### Experimental Vaccine Study

The objectives of the study were initially to test the ability a DNA vaccine of the invention to induce neo-peptide specific T cells and to monitor the impact of the vaccine on the well-being of vaccinated mice.

Plasmids for DNA vaccination were based on the commercially available pVax1™ vector available from ThermoFisher Scientific/Invitrogen.

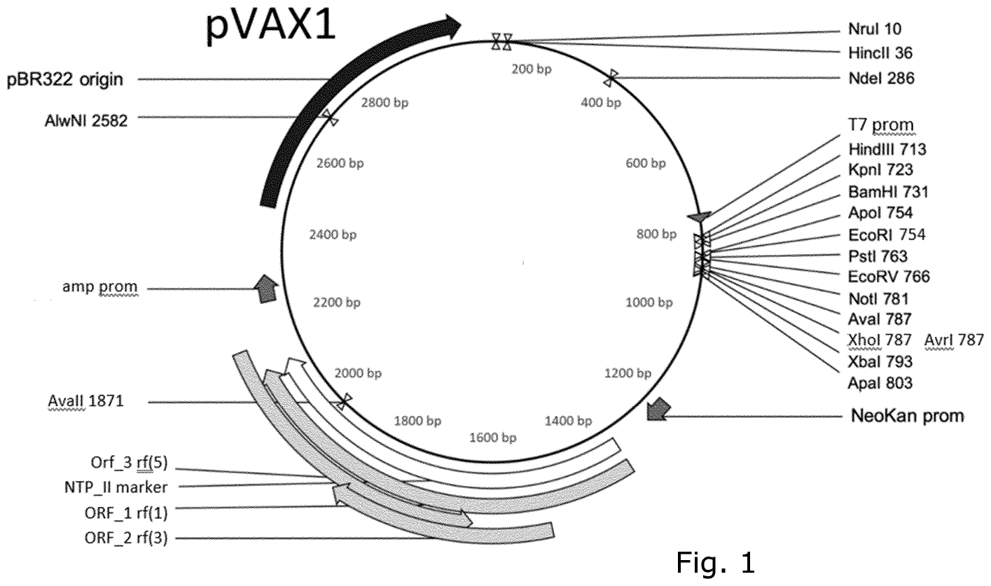

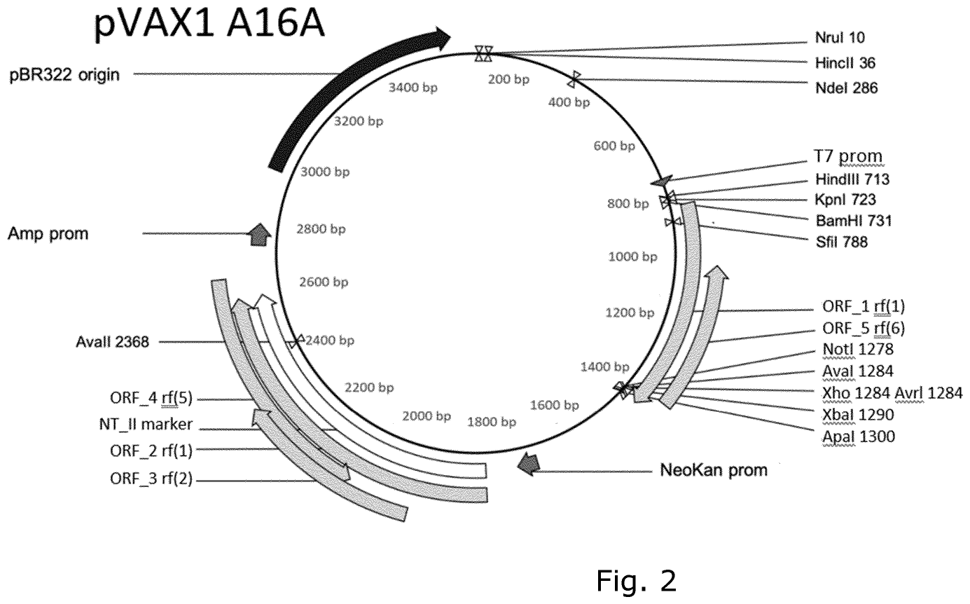

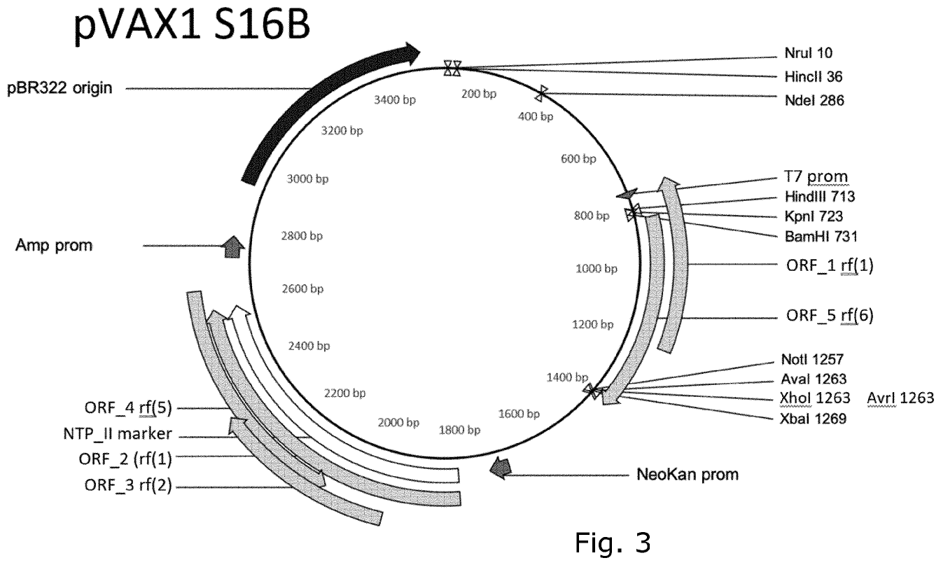

pVAX1™ is according to the manufacturer's documentation a 3.0 kb plasmid vector, which allows high-copy number replication in E. coli and high-level transient expression of encoded protein of interest in most mammalian cells. The vector (see FIG. 1) contains the following elements:

1) a human cytomegalovirus immediate-early (CMV) promoter for high-level expression in a wide range of mammalian cells,

2) a bovine growth hormone (BGH) polyadenylation signal for efficient transcription termination and polyadenylation of mRNA, and

3) A kanamycin resistance gene for selection in E. coli.

The entire sequence of the pVAX1™ plasmid is set forth in SEQ ID NO: 1. For use as a positive control for transfection and expression in the cell line of choice, one can use the control plasmid, pVAX1™/lacZ, the sequence of which is set forth in SEQ ID NO: 2.

Two expression vectors, pVAX1 S16A and pVAX1 S16B were constructed.

Neo-peptides/neo-epitopes were first identified by whole exome sequencing of the mouse colon cancer cell line CT26 and normal tissue samples from BALB/c mice and by selecting peptides found only in the cancer cells as evidenced by measuring RNA expression levels. In the experiment, the ability of the mice to generate immune responses against the identified neo-epitopes was evaluated.

pVAX1 S16A was constructed by ligating codon-optimized (for expression in mice) DNA encoding a peptide containing the sequentially coupled 5 neo-epitopes C22, C23, C25, C30, and C38 (SEQ ID NOs: 11-15) into the pVax1 expression cassette. pVax1 S16B was similarly constructed by ligating codon-optimized DNA encoding the sequentially coupled neo-epitopes C29, C37, C39, C40, and C41 (SEQ ID NOs: 16-20) into the pVax1 expression cassette. For use as control, a peptide mixture of C22, C23, C25, C26 (SEQ ID NO: 21), and C38 formulated in the CAF09 adjuvant discussed above. In the pVAX1 S16A and S16B vectors, the inserts also included a Kozak consensus sequence to effectively initiate translation. The 11 amino acid sequences used in the experiments (also in the following examples) are set forth in the following table:

| Peptide | AA Sequence | SEQ ID NO: |
|---|---|---|
| C22 | QIETQQRKFKASRASILSEMKMLKEKR | 11 |
| C23 | VILPQAPSGPSYATYLQPAQAQMLTPP | 12 |
| C25 | DTLSAMSNPRAMQVLLQIQQGLQTLAT | 13 |
| C30 | DGQLELLAQGALDNALSSMGALHALPR | 14 |
| C38 | RLHVVKLLASALSTNAAALTQELLVLD | 15 |
| C29 | LHSGQNHLKEMAISVLEARACAAAGQS | 16 |
| C37 | GEVPPQKLQALQRALQSEFCNAVREVY | 17 |
| C39 | KKFMERDPDELRFNTIALSA | 18 |
| C40 | VTGTHKMSLGFTKARLLRLRNPWGRVE | 19 |
| C41 | LWTFSIYLESVAIMPQLFMVSKTGEAE | 20 |
| C21/C26 | GDVKIHAHKVVLANISPYFKAMFTGNL | 21 |
| EV85 | KKFMERDPDELRFNTIALSAA | 43 |
| EV22 | GSLFGSSRVQYVVNPAVKIVFLNIDPS | 44 |
| EV105 | PPPGLAAYTAKMATANGSKKAERQKFS | 45 |
| AA427 | VCNVKLLHRVLVADVNALQGMAAIGQR | 46 |

Plasmids pVAX1 516A, pVAX1 516B, and (empty) pVAX1 solubilised in sterile water were each mixed with poloxamer 188 (Lutrol® F68 from Sigma Aldrich) and Tyrode's buffer to obtain a composition of 3% v/v poloxamer 188 and 1 μg/μl plasmid in Tyrode's buffer. A vehicle solution of water, Tyrode's buffer (in the ratio water:buffer of and poloxamer 188 (3% v/v) was also prepared.

In parallel, the above-described peptide mixture was prepared by mixing DMSO solubilised peptides, Tris buffer and CAF09 to provide for a composition comprising 5% w/v of each peptide (i.e. 25% w/v of total peptides), 5% DMSO, 66.67% v/v CAF09 in Tris buffer.

One group of 5 mice was immunized with the peptide composition at days 0, 1, 2, 3, 9, and 16 at dosages of 50 μg of each peptide. In parallel, 4 groups of 5 mice received injections of 100 μl of DNA vaccine (or mock control) in the form of 50 μl in each tibia at days 2, 9 and 16. The four groups were immunized with poloxamer 188 only, poloxamer 188 and mock plasmid, poloxamer 188 and pVAX1 S16A, and poloxamer 188 and pVAX1 S16B, respectively.

To gauge T-cell activation, the following re-stimulation experiment was carried out:

Splenocytes were stimulated with vaccine-containing neo-peptides. In the splenocyte samples, antigen presenting cells process the neo-peptides and subsequently present them to T cells, leading to activation of cognate CD4⁺ and CD8⁺ T cells. The activated T cells increase cytokine synthesis, including tumour necrosis factor α (TNF-α) and interferon γ (IFN-γ). Multifunctional T cells were detected by staining with cytokine and cell surface marker specific fluorochrome labelled antibodies.

### Results

Whole blood from all mice where collected at day 13 post first immunization and stained with fluorophore labelled C22 MHC I tetramers.

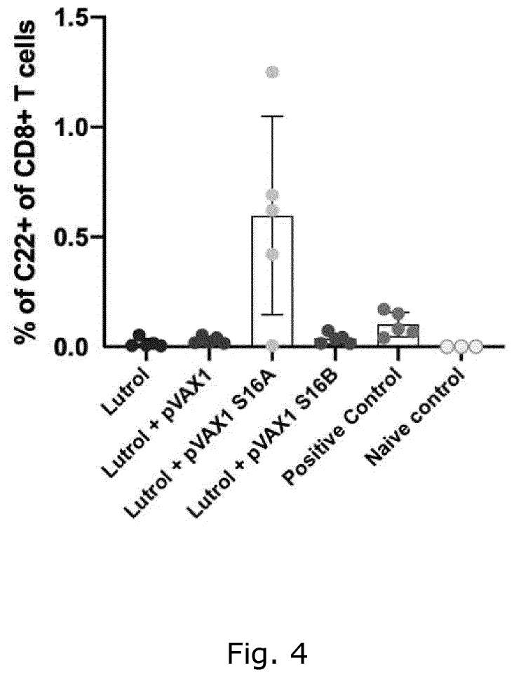

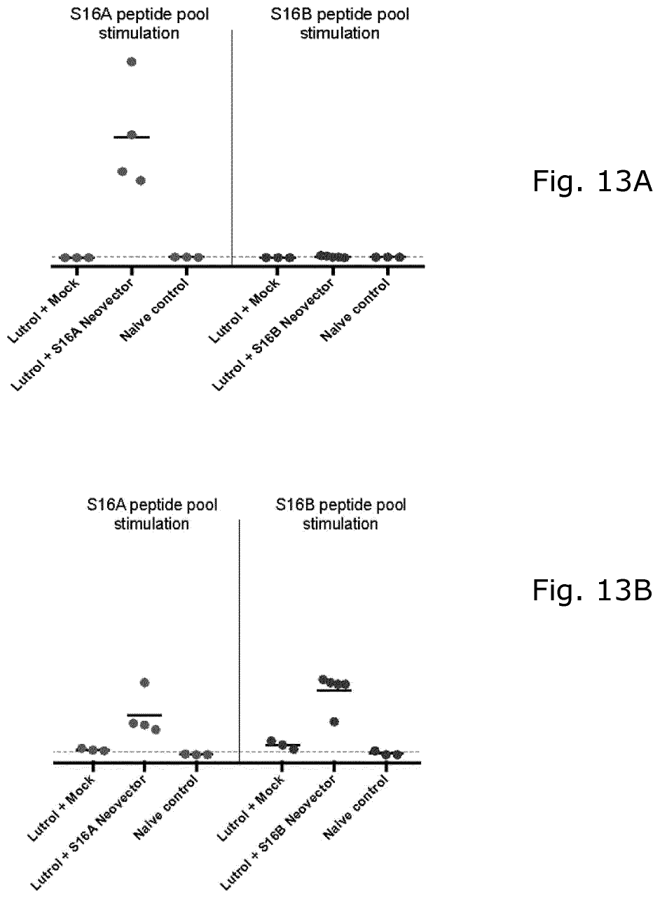

Immunizations day 0 and 7 with the C22 encoding pVAX1 S16A vaccine induced C22 neo-peptide specific CD8⁺ T cells at high frequencies (average 0.6 frequency). See FIG. 4. The effect was markedly better than the positive control (average 0.1 frequency) consisting of five peptides (including C22) formulated in CAF09. The vaccination with the S16A plasmid induced CD8⁺ T cells capable of producing IFNγ and TNFα in response to subsequent stimulation with neo-peptides, whereas samples from animals not immunized with S16A exhibited no cytokine signals upon stimulation with the neo-peptides. Further, vaccination with S16A and S16B induced reactive CD4⁺ T cells capable of producing IFNγ and TNFα in response to subsequent stimulation with neo-peptides, whereas samples from animals not immunized with S16A exhibited no cytokine signals upon stimulation with the neo-peptides. Data for the two experiments are shown in FIGS. 13A and 13B.

No C22 neo-peptide specific CD8⁺ T cells were detected in any of the mice from the remaining groups.

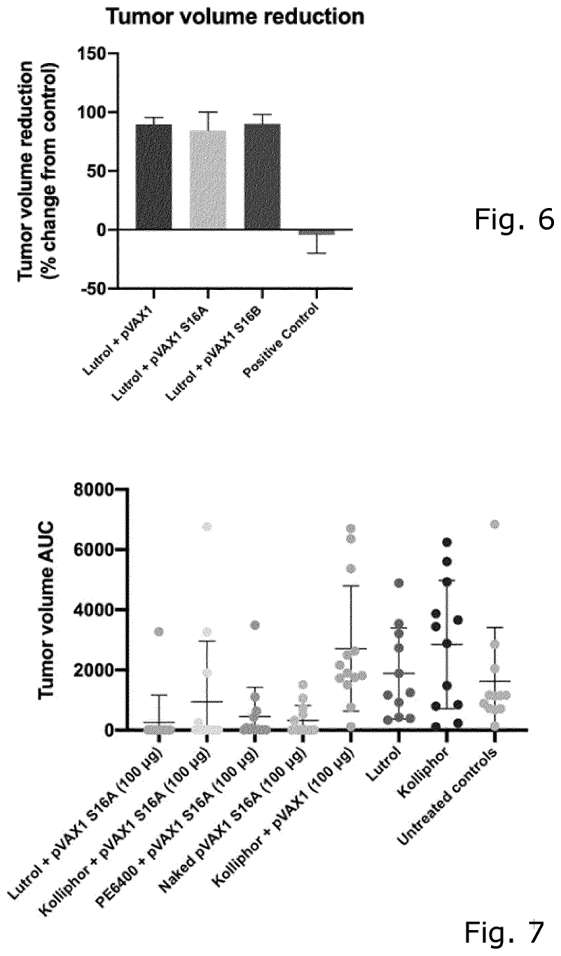

In a pilot add-on experiment, mice were immunized with the plasmid vaccines as indicated above. 4 weeks after the first immunization (i.e. between immunizations 4 and 5, tumour cells (CT26) were inoculated. After 43 days, the mice were sacrificed and tumour sizes determined. This pilot experiment exhibited complete eradication of the transplanted tumours in the majority of the mice in the groups receiving plasmid vaccines. See FIG. 6. The study was not powered to obtain statistically significant data, since only 5 mice per group was immunized. The dose of 100 μg DNA was consequently too high to discriminate between the effect induced by DNA mediated TLR9 engagement and neo-epitope specific T cells, thus calling for an experiment with reduction of the DNA plasmid dose and/or the number of immunizations. Further, the DNA vaccines were well-tolerated by the mice; no signs of adverse effects were observed and the body weight of the mice continuously increased throughout the study, indicative of healthy and unaffected mice.

### Conclusions

In whole blood from plasmid vector S16A vaccinated mice, C22 neo-peptide specific CD8⁺ T cells were present at high frequencies. Based of these data it was decided to inoculate the mice with CT26 tumour cells and continue the 7 days immunization schedule. Immunizations with 7 days interval starting 4 weeks prior to tumour cells inoculation, resulted in complete eradication of the transplanted tumours in the majority of the DNA plasmid vaccinated mice The immunization schedule of 100 μg DNA plasmid was too intensive to discriminate between the effect induced by DNA mediated TLR9 engagement and neo-epitope specific T cells, thus calling for a reduction of the number of immunizations. The plasmid vector S16A vaccine gave rise to CD8⁺ T cell neo-peptide reactivity, measured by cytokine production upon stimulation, whereas the plasmid vector pVAX1 S16B vaccine predominantly induced a CD4⁺ T cells response and not a CD8⁺ T cells response. The vaccines were well-tolerated by the mice; no signs of adverse effects were observed, and the body weight of the mice continuously increased throughout the study, indicative of healthy and unaffected mice.

### Example 2

### Assessment of Plasmid Vector +/− Different Polymers in a CT26 Tumour Study

The objectives of the study were to investigate the immunogenicity and anti-tumour effects of the S16A Plasmid vector delivered as a naked DNA vaccine or in combination with a selection of different block co-polymers.

### Study Plan

Mice received immunizations with the test vaccines on days −13, −6, 1, 7, and 14 relative to the CT26 tumour inoculation on day 0. Each immunization consisted of injection of 50 μl vaccine in the left and right tibia, respectively. Blood samples for C22 MHC I testing in a tetramer assay were obtained from the test animals on day 1 after inoculation.

The tetramer assay was carried out as follows:

MHC class I molecules are produced and loaded with a stabilizing peptide that is exchanged with the C22 epitope by exposing the molecules to UV light. The MHC I molecules are multimerized by coupling to fluorescently labelled Streptavidin. To identify neo-peptide positive CD8⁺ T cells, cells are co-stained with the multimers and fluorophore conjugated anti-CD3, anti-CD4 and anti-CD8 antibodies. Samples are then analyzed by flow cytometry and the fraction of MHC:C22 positive CD8⁺ is calculated

8 groups of 13 mice received the following vaccine compositions respectively:
1. Lutrol+S16 A Plasmid vector (100 μg)
2. Kolliphor+S16 A Plasmid vector (100 μg)
3. PE6400+S16 A Plasmid vector (100 μg)
4. Kolliphor+Mock Plasmid vector (100 μg)
5. Naked S16 A Plasmid vector (100 μg)
6. Lutrol
7. Kolliphor
8. Untreated controls

A ninth group of naive mice included 5 animals.

The amphiphilic block co-polymers tested in combination with the S16A were thus Lutrol® F 68 and Kolliphor® P188, both of the general formula:

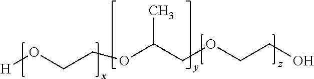

Also tested was PE6400® (BASF) with the formula:

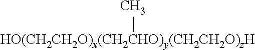

The plasmid vector vaccines consist of the above polymers formulated with 100 μg plasmid DNA in the form of the PVAX1, S16A, and the Mock plasmid:

| | | | Vaccine components | | | |
|---|---|---|---|---|---|---|
| Study | | DNA | Vol. | H₂O | Tyrode's | Polymer |
| group | Treatment | dose (μg) | plasmid (μl) | (μl) | buffer (μl) | (μl) |
| 1 | Lutrol + 16SA | 100 | 742.9 | 0 | 349.1 | 468 |
| 2 | Kolliphor + 16SA | 100 | 742.9 | 0 | 349.1 | 468 |
| 3 | PE6400 + 16SA | 100 | 742.9 | 390.0 | 349.1 | 78 |
| 4 | Kolliphor + Mock DNA | 100 | 433.3 | 309.5 | 349.1 | 468 |
| 5 | S16A DNA | 100 | 742.9 | 468.0 | 349.1 | 0 |
| 6 | Lutrol | 0 | 0 | 742.9 | 349.1 | 468 |
| 7 | Kolliphor | 0 | 0 | 742.9 | 349.1 | 468 |

Read-outs of the experiment were body weight change relative to the weight at the first immunization, tumour volume, evaluation of T-cell activation upon peptide re-stimulation, and measurement of neo-epitope-specific CD8⁺ T cells in circulation.

### Results

The effect on tumour growth of the immunizations is shown in FIG. 7: Prophylactic immunizations resulted in significantly lower tumour volume for mice receiving 100 μg S16A plasmid vector with co-polymers Kolliphor, Lutrol and PE6400 as well as naked S16A plasmid DNA, compared to 100 μg mock plasmid vector and co-polymer only delivery.

Co-polymer facilitated delivery of S16A plasmid vector did not result in significantly lower tumour volume than naked S16A plasmid vector. Somewhat lower tumour volumes than expected were observed in Lutrol only and Untreated control groups, albeit significantly larger tumour volume than 100 μg S16A naked plasmid delivery. The effects on tumour volume were also confirmed in mice followed for longer periods, where the untreated controls all developed tumours whereas between 60 and 80% of the vaccinated animals

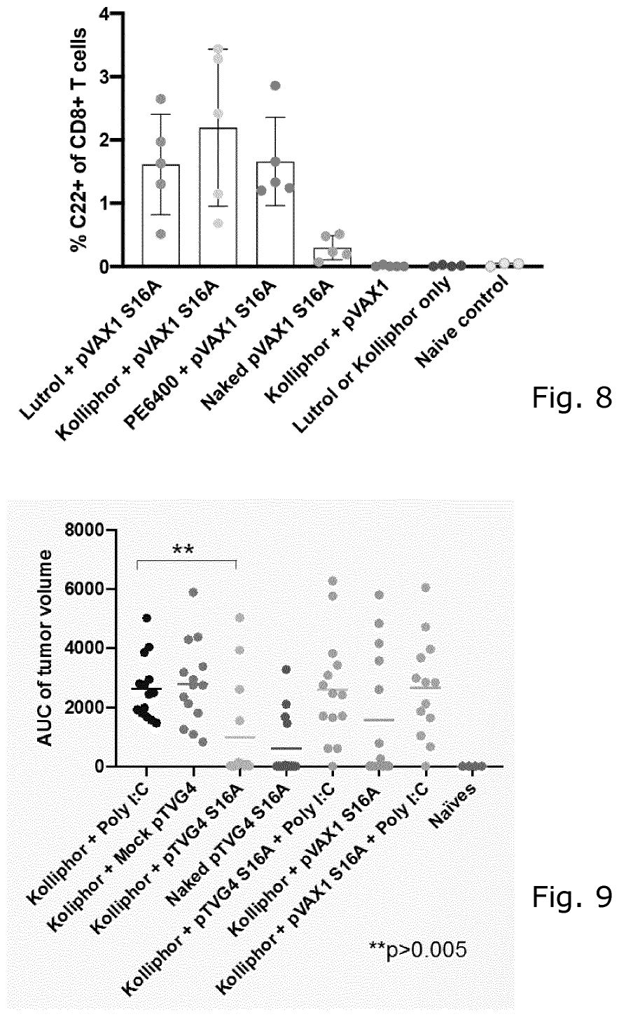

In addition (see FIG. 8) the copolymer containing vaccines provided for an earlier onset of immune responses. This was observed as the emergence of CD8⁺ cells when testing for their presence in a C22 tetramer staining assay: CD8⁺ T cells specific to the neo-peptide C22 were observed in tail vein blood from study day 1 (one day after the third immunization) in mice immunized with S16A plasmid vector with and without co-polymers, but significantly higher frequency of CD8⁺ T cells were observed at this time point for groups receiving the copolymer formulated vaccines. There was no observable difference between Lutrol, Kolliphor and PE6400 co-polymers to facilitate S16A immunogenicity

No tetramer signal was detected in control samples

### Conclusions

The S16A plasmid vector delivery with and without different block co-polymers resulted in CT26 anti-tumour effects. 00 μg S16A vector (with Lutrol, Kolliphor, PE6400, and as naked DNA) resulted in significantly lower tumour volume AUC compared to control groups, demonstrating that clinical grade polymer Kolliphor is as efficacious as the research grade version (Lutrol). There was no anti tumour effect from 100 μg mock plasmid immunization. Co-polymer facilitated plasmid vector delivery was more immunogenic early on in the treatment than naked plasmid vector DNA. S16A neo-peptide re-stimulation showed similar T cell immunogenicity profiles in splenocytes across groups that received S16A Plasmid vector with or without co-polymer.

### Example 3

### Test of Candidate Vaccine

The main objective of this example was to test the vector pTVG4 as a backbone for delivering neo-epitopes encoded by a plasmid. The pTVG4 vector containing the pentatope S16A predicted for the CT26 mouse tumour model (see above for details) was tested and benchmarked vs. the pVAX1 backbone with the S16A pentatope.

Furthermore a secondary objective was to test the TLR3 agonist Poly I:C in combination with a DNA vaccine to determine if the combination resulted in a higher neo-epitope specific T cell number.

### Experimental

Mice received immunizations with the test vaccines on days −13, −6, 1, 7, and 14 relative to the CT26 tumour inoculation on day 0. Each immunization consisted of injection of 50 μl vaccine in the left and right tibia, respectively. Blood samples for C22 MHC I testing in a tetramer assay were obtained from the test animals on day 1 after inoculation.

7 groups of 13-15 mice received the following vaccine compositions respectively:
1. Kolliphor+Poly I:C
2. Kolliphor+Mock pTVG4
3. Kolliphor+pTVG4 S16A
4. Naked pTVG4 S16A
5. Kolliphor+pTVG4 S16A+poly I:C
6. Kolliphor+pVAX1 S16A
7. Kolliphor+pVAX1 S16A+poly I:C

An 8^{th} group of 4 naive mice was also tested.

Read-outs were tumour volume, splenic T-cell activation upon peptide re-stimulation and neo-epitope specific CD8⁺ T-cells in circulation.

### Results

The effect on tumour sizes is set forth in FIG. 9. Immunizations with 50 μg pTVG4a S16A+Kolliphor and 50 μg pVAX1 S16A+Kolliphor both resulted in retardation of CT26 tumour growth for the majority of mice. The naked pTVG4a S16A vector had a similar effect. Somewhat surprisingly, co-formulation of poly I:C with pTVG4a S16A and pVAX1 S16A counteracted the antitumour effect of the two DNA immunotherapies.

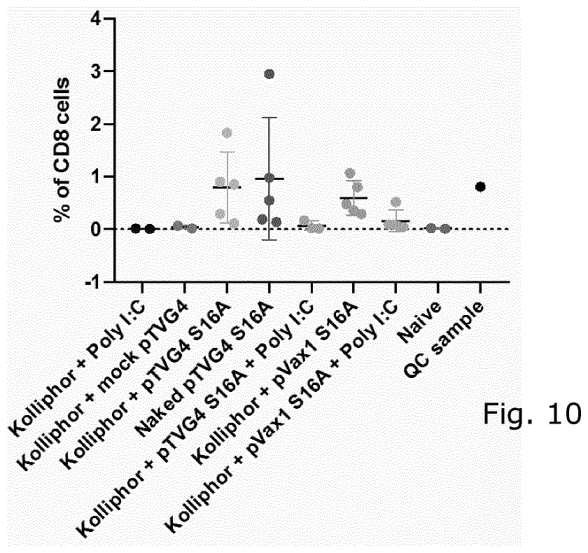

A similar pattern appears from FIG. 10, which shows the C22 tetramer staining of blood obtained on day 6 after inoculation: CD8⁺ T-cells specific to CT26 neo-peptide C22 were observed in tail vein blood at study day 6 in mice immunized with pTVG4a S16A +/−Kolliphor and pVAX1 S16A+Kolliphor, but there was a lower frequency of C22-specific CD8⁺ T cells observed at in groups receiving poly I:C together with the DNA immunotherapy. As expected no tetramer signal was detected in control samples (Kolliphor+poly I:C immunized or naive control mice).

### Conclusions

The pTVG4 vector successfully delivered encoded neo-epitopes and induced a specific T cell response and also reduced the tumour volume to a at least a similar extent as the previously tested pVAX1 S16 plasmid. The combination with poly I:C did in this experiment appeared to counteract the anti-tumour effect of the tested vaccines.

### Example 4

### Test of an Optimized DNA Insert Sequence

The objective was to determine whether the expression of different neo-epitopes is affected by their position within the insert in the vaccine vector and if this translates into differences in the antitumour effect (positional bias testing).

Further the objective was to test a clinically relevant DNA construct harboring 13 neo-epitopes and compare to the pentatope tested above.

### Experimental

7 groups of 14 mice were used in an experiment where 5 groups received test vaccines (50 μg DNA in combination with Kolliphor®), one group received an empty vector, and group received adjuvant only. Further, a group of 5 naive mice was also included in the experiment. Immunizations were given on days −15, −8, −1, 6, and 13 relative to inoculation with CT26 tumour cells on day 0. On day −2, blood was sampled.

The group and vaccine allocation was as follows:

| Group/treatment | Description |
|---|---|
| Kolliphor | Adjuvant only |
| Kolliphor + Mock pTGV4 | Empty vector |
| Kolliphor + pTGV4 S16A | 5 neo-epitopes, non-optimized linker sequence |
| Kolliphor + pTGV4 S16A OPTIM | 5 neo-epitopes, optimized linker sequence |
| Kolliphor + pTGV4 S16T13 F/OPTIM | 13 neo-epitopes, optimized linker sequence |
| Kolliphor + pTGV4 S16T13 M/OPTIM | 13 neo-epitopes, optimized linker sequence |
| Kolliphor + pTGV4 S16T13 R/OPTIM | 13 neo-epitopes, optimized linker sequence |
| Na\ddot{ι}ve mice | N/A |

The “OPTIM” designation indicates that the insert DNA sequence was generated with a codon optimized vector sequence. For positional bias testing, one specific neo-epitope, C22, was moved from first, to middle to end position of a 13 neo-epitope encoding insert, named S16T13 F/OPTIM, S16T13 M/OPTIM and R/OPTIM—this approach was used to determine whether the entire DNA insert is indeed expressed and whether the relative position of the epitopes has an influence on expression efficacy. The rationale for choosing C22 as the moving epitope relates to the ease of detection in the established C22 specific CD8⁺ T cells detection with the MHC I tetramer assay.

The S16T13 polyepitope included the 13 epitopes C22, C23, C38, C25, C30, C37, EV85, C40, C41, C29, EV22, EV105, and AA427.

As in the previous experiments, read-outs were tumour volume (on day 19), splenic T-cell activation upon peptide re-stimulation and neo-epitope specific CD8⁺ T-cells in circulation.

### Results

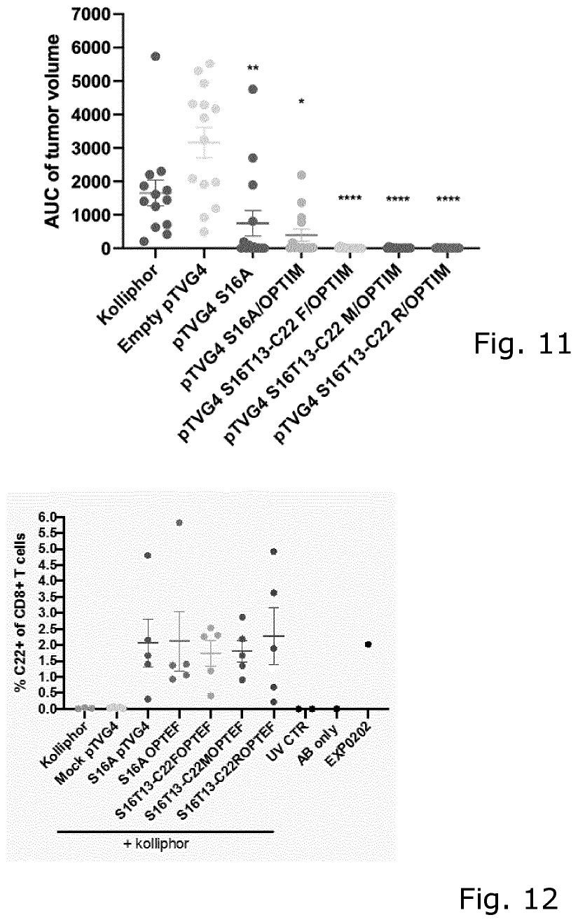

The vaccines' effects on tumour volume (on day 19) and CD8⁺ T-cells specific for C22 (on day −2) are shown in FIGS. 11 and 12, respectively.

For the tumour volumes, a Kruskal-Wallis test evidenced that all 13-neo-epitope constructs reduced tumour growth completely (p<<0.001), while both 5-neo-epitope constructs also reduced tumour growth significantly. With respect to CD8⁺ T-cells recognizing C22, all constructs induced a similar degree of CD8⁺ T-cell recognition, thus confirming that the entire DNA insert is transcribed and translated in vivo.

### Conclusion

All tested nucleotide sequences efficiently induced a neo-epitope specific T cell response translating into anti-tumour immunity. The position of the neo-epitopes within the DNA insert did not affect the immunogenicity: Similar frequencies of neo-epitope specific CD8⁺ T cells were induced by all construct regardless the position of the measured neo-epitope (C22).

Finally, and important, the number of included neo-epitopes impacted the antitumour effect of the DNA construct. Inclusion of more neo-epitopes (5 vs. 13) improved the clinical response significantly.

1. A method of inducing a therapeutic or ameliorating immune response against a malignant neoplasm in a patient, wherein the cells of the malignant neoplasm express genetic material that encode neo-epitope containing polypeptides, the method comprising administering to the patient at least one effective dosage of a composition comprising
1) at least one expression vector, which comprises nucleic acid(s) encoding at least one polypeptide, which exhibits one or more neo-epitopes of the malignant neoplasm, and
2) an amphiphilic block co-polymer comprising blocks of poly(ethylene oxide) and polypropylene oxide), and
3) a pharmaceutically acceptable carrier, diluent, or excipient, whereby somatic cells in the patient are brought to express the nucleic acid(s) encoding the at least one polypeptide.

2. The method according to claim 1, wherein the amphiphilic block co-polymer is a poloxamer or a poloxamine.

3. The method according to claim 1, wherein amphiphilic block co-polymer is a poloxamer selected from poloxamer 407 and 188, preferably poloxamer 188.

4. The method according to claim 1, wherein the amphiphilic block co-polymer is a sequential poloxamine of formula (PEO-PPO)₄-ED, where PEO is poly(ethylene oxide), PPO is poly(propylene oxide) and ED is an ethylenediaminyl group.

5. The method according to any one of the preceding claims, wherein the pharmaceutically acceptable carrier, diluent, or excipient is an aqueous buffered solution.

6. The method according to claim 5, wherein the aqueous buffered solution is Tyrode's buffer or 2-amino-2-(hydroxymethyl)-1,3-propandiol (TRIS) buffer or PBS.

7. The method according to claim 6, wherein the Tyrode's buffer has the composition 140 mM NaCl, 6 mM KCl, 3 mM CaCl₂, 2 mM MgCl₂, 10 mM 4-(2-hydroxyethyl)-1-piperazineethanesulfonic acid (Hepes), and 10 mM glucose, or has the composition 140 mM NaCl, 6 mM KCl, 3 mM CaCl₂, 2 mM MgCl₂, 10 mM 2-amino-2-(hydroxymethyl)-1,3-propandiol (TRIS), and 10 mM glucose.

8. The method according to any one of the preceding claims wherein the concentration of Tyrodes' buffer is about 35% v/v.

9. The method according to any one of the preceding claims wherein the concentration of the amphiphilic block co-polymer is between 2 and 5% w/v, such as about 3% w/v.

10. The method according to any one of the preceding claims, wherein the composition further comprises at least one immune stimulating sequence (ISS) .

11. The method according to claim 10, wherein the ISS is an oligodeoxyribonucleotide (ODN) comprising at least one CpG motif, and wherein the ODN preferably includes phosphorothioate groups.

12. The method according to claim 10, wherein the ISS is or comprises an oligoribonucleotide.

13. The method according to any one of the preceding claims, wherein the effective dosage contains between 10 μg and 25 mg of the expression vector, such as between 100 μg and 20 mg, between 0.5 and 15 mg, between 1 and 10 mg, and between 2 and 8 mg, in particular about 0.5, about 1, about 2, about 3, about 4, about 5, about 6, about 7 and about 8 mg.

14. The method according to any one of the preceding claims wherein the expression vector expresses at least or about 5, such as at least or about 10, at least or about 15, at least or about 20, at least or about 25, and at least or about 30 neo-epitopes.

15. The method according to any one of the preceding claims, wherein the expression vector encodes a separate peptide for each encoded neo-epitope.

16. The method according to any one of the preceding claims, wherein the expression vector encodes a plurality of peptides, where at least one exhibit(s) several encoded neo- epitopes, of which at least some optionally are separated by peptide linkers.

17. The method according to any one of the preceding claims, wherein the expression vector further comprises or encodes at least one Immune Stimulatory Sequence (ISS).

18. The method according to claim 17, wherein the at least one ISS or ISS encoding sequence is/are positioned between the stop codon of a neo-epitope encoding sequence and a polyadenylation signal.

19. The method according to any one of claims 17-18, wherein the at least one ISS is comprised in the expression vector.

20. The method according to any one or claims 17-18, wherein the at least one ISS encoding sequence is encoded by the expression vector.

21. The method according to claim 19, wherein the ISS is or comprises a sequence that activates Toll-like receptor 9 (TLR-9), such as a CpG motif.

22. The method according to claim 20, wherein the ISS encodes an RNA sequence that activates Toll-like receptor 3 (TLR-3) and/or cytosolic RNA receptors such as RIG-1, MDA5, and LGP2, such as an RNA sequence the forms an RNA hairpin or constitute an immune stimulating RNA sequence.

23. The method according to any one of the preceding claims, wherein the expression vector is comprised in or constitutes a plasmid.

24. The method according to any one of the preceding claims, wherein the at least one effective dosage is a series of dosages, such as a series of 2, 3, 4, 5, 6, or more dosages.

25. The method according to any one of the preceding claims, wherein the patient is a human being.

26. The method according to any one of the preceding claims, wherein the effective dose is administered parenterally, such as via the intramuscular route, the intradermal route, transdermal route, the subcutaneous route, the intravenous route, the intra-arterial route, the intratechal route, the intramedullary route, the intrathecal route, the intraventricular route, the intraperitoneal, the intranasal route, the vaginal route, the intraocular route, or the pulmonary route; is administered via the oral route, the sublingual route, the buccal route, or the anal route; or is administered topically.

27. A composition comprising
1) at least one expression vector, which comprises nucleic acid(s) encoding at least one polypeptide, which exhibits one or more neo-epitopes of a malignant neoplasm, and
2) an amphiphilic block co-polymer comprising blocks of poly(ethylene oxide) and polypropylene oxide), and
3) Tyrode's buffer.

28. The composition according to claim 27, which is as the composition defined in any one of claims 2-23.

29. A composition as defined in any one of claims 1-23 for use as a medicament.

30. A composition as defined in any one of claims 1-23, for use in a method according to any one of claims 1-14.


SEQUENCE LISTING (46 sequences)

SEQ ID NO: 1 - DNA, 2999 bp, Artificial sequence
  pVAX1 DNA vaccine vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 2 - DNA, 6050 bp, Artificial sequence
  pVAX1/LacZ DNA vaccine vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatcgaaacgatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 3 - protein, 6 aa, Artificial sequence
  Flexible linker peptide
GSGGGA

SEQ ID NO: 4 - protein, 12 aa, artificial sequence
  Flexible linker peptide
GSGGGAGSGGGA

SEQ ID NO: 5 - protein, 18 aa, Artificial sequence
  Flexible linker peptide
GSGGGAGSGGGAGSGGGA

SEQ ID NO: 6 - protein, 24 aa, Artificial sequence
  Flexible linker peptide
GSGGGAGSGGGAGSGGGAGSGGGA

SEQ ID NO: 7 - protein, 10 aa, Artificial sequence
  Flexible linker peptide
GENLYFQSGG

SEQ ID NO: 8 - protein, 12 aa, Artificial sequence
  Rigid linker peptide
KPEPKPAPAPKP

SEQ ID NO: 9 - protein, 12 aa, Artificial sequence
  Rigid linker peptide
AEAAAKEAAAKA

SEQ ID NO: 10 - protein, 8 aa, Artificial sequence
  Rigid linker peptide
SACYCELS

SEQ ID NO: 11 - protein, 27 aa, Artificial sequence
  Neo-epitope
QIETQQRKFKASRASILSEMKMLKEKR

SEQ ID NO: 12 - protein, 27 aa, Artificial sequence
  Neo-epitope
VILPQAPSGPSYATYLQPAQAQMLTPP

SEQ ID NO: 13 - protein, 27 aa, Artificial sequence
  Neo-epitope
DTLSAMSNPRAMQVLLQIQQGLQTLAT

SEQ ID NO: 14 - protein, 27 aa, Artificial sequene
  Neo-epitope
DGQLELLAQGALDNALSSMGALHALPR

SEQ ID NO: 15 - protein, 27 aa, Artificial sequence
  Neo-epitope
RLHVVKLLASALSTNAAALTQELLVLD

SEQ ID NO: 16 - protein, 27 aa, Artificial sequence
  Neo-epitope
LHSGQNHLKEMAISVLEARACAAAGQS

SEQ ID NO: 17 - protein, 27 aa, Artificial sequence
  Neo-epitope
GEVPPQKLQALQRALQSEFCNAVREVY

SEQ ID NO: 18 - protein, 20 aa, Artificial sequence
  Neo-epitope
KKFMERDPDELRFNTIALSA

SEQ ID NO: 19 - protein, 27 aa, Artificial sequence
  Neo-epitope
VTGTHKMSLGFTKARLLRLRNPWGRVE

SEQ ID NO: 20 - protein, 27 aa, Artificial sequence
  Neo-epitope
LWTFSIYLESVAIMPQLFMVSKTGEAE

SEQ ID NO: 21 - protein, 27 aa, Artificial sequence
  Neo-epitope
GDVKIHAHKVVLANISPYFKAMFTGNL

SEQ ID NO: 22 - protein, 10 aa, Artificial sequence
  Peptide linker
SGGGSSGGGS

SEQ ID NO: 23 - protein, 10 aa, Artificial sequence
  Peptide linker
GGGGSGGGGS

SEQ ID NO: 24 - protein, 10 aa, Artificial sequence
  Peptide linker
SSGGGSSGGG

SEQ ID NO: 25 - protein, 10 aa, Artificial sequence
  Peptide linker
GGSGGGGSGG

SEQ ID NO: 26 - protein, 10 aa, Artificial seuence
  Peptide linker
GSGSGSGSGS

SEQ ID NO: 27 - DNA, 20 bp, Artificial sequence
  CpG motif
ggggtcaacgttgagggggg

SEQ ID NO: 28 - DNA, 20 bp, Artificial sequence
  CpG motif
gggggacgatcgtcgggggg

SEQ ID NO: 29 - DNA, 21 bp, Artificial sequence
  CpG motif
ggggacgacgtcgtggggggg

SEQ ID NO: 30 - DNA, 20 bp, Artificial sequence
  CpG motif
tccatgacgttcctgatgct

SEQ ID NO: 31 - DNA, 20 bp, Artificial sequence
  CpG motif
tccatgacgttcctgacgtt

SEQ ID NO: 32 - DNA, 24 bp, Artificial sequence
  CpG motif
tcgtcgttttgtcgttttgtcgtt

SEQ ID NO: 33 - DNA, 22 bp, Artificial sequence
  CpG motif
tcgtcgttgtcgttttgtcgtt

SEQ ID NO: 34 - DNA, 23 bp, Artificial sequence
  CpG motif
tcgacgttcgtcgttcgtcgttc

SEQ ID NO: 35 - DNA, 26 bp, Artificial sequence
  CpG motif
tcgcgacgttcgcccgacgttcggta

SEQ ID NO: 36 - DNA, 22 bp, Artificial sequence
  CpG motif
tcgtcgttttcggcgcgcgccg

SEQ ID NO: 37 - DNA, 25 bp, Artificial sequence
  CpG motif
tcgtcgtcgttcgaacgacgttgat

SEQ ID NO: 38 - DNA, 29 bp, Artificial sequence
  CpG motif
tcgcgaacgttcgccgcgttcgaacgcgg

SEQ ID NO: 39 - DNA, 75 bp, Artificial sequence
  Fragment of pTVG4 vaccine vector
agatctaacgacaaaacgacaaaacgacaaggcgccagatctggcgtttcgttttgtcgttttgtcgttagatct

SEQ ID NO: 40 - DNA, 4099 bp, Artificial sequence
  pTVG4 vaccine vector
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctaacgacaaaacgacaaaacgacaaggcgccagatctggcgtttcgttttgtcgttttgtcgttagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 41 - DNA, 20 bp, Artificial sequence
  CPG encoding sequence
ggtgcatcgatgcagggggg

SEQ ID NO: 42 - DNA, 83 bp, Artificial sequence
  CPG encoding sequence
ggtgcatcgatgcaggggggtatatatatattgaggacaggttaagctccccccagcttaacctgtccttcaatatatatata

SEQ ID NO: 43 - protein, 21 aa, Artificial sequence
  Neo-epitope
KKFMERDPDELRFNTIALSAA

SEQ ID NO: 44 - protein, 27 aa, Artificial sequence
  Neo-epitope
GSLFGSSRVQYVVNPAVKIVFLNIDPS

SEQ ID NO: 45 - protein, 27 aa, Artificial sequence
  Neo-epitope
PPPGLAAYTAKMATANGSKKAERQKFS

SEQ ID NO: 46 - protein, 27 aa, Artificial sequence
  Neo-epitope
VCNVKLLHRVLVADVNALQGMAAIGQR